(19) United States
(12) Patent Application Publication
(10) Pub. No.: US 2026/0105601 A1
(43) Pub. Date: Apr. 16, 2026

(54) PRIMARY AUDITORY CORTEX REGION SEGMENTING DEVICE AND METHOD

(71) Applicant: Korea University Research and Business Foundation, Seoul (KR)

(72) Inventor: Bo-Yong PARK, Seoul (KR)

(21) Appl. No.: 19/331,831

(22) Filed: Sep. 17, 2025

(30) Foreign Application Priority Data
Oct. 16, 2024 (KR) 10-2024-0141648

(51) Int. Cl. G06T 7/00; G06T 7/12; G06T 7/13; G06V 10/25; G06V 10/44; G06V 10/762; G16H 30/20; G16H 30/40

(52) U.S. Cl. CPC G06T 7/0012; G06T 7/12; G06T 7/13; G06V 10/25; G06V 10/44; G06V 10/762; G16H 30/20; G16H 30/40; G06T 2207/10088; G06T 2207/30016

(57) ABSTRACT

The present disclosure relates to a primary auditory cortex region segmenting device for segmenting a primary auditory cortex which is responsible for auditory perception, including: an acquisition circuitry which collects multimodal magnetic resonance image data and acquires a myelin feature image and a curvature feature image from the multimodal magnetic resonance image data; a region of interest setting circuitry which sets an initial region of interest for segmenting the auditory cortex in the curvature feature image based on a general-purpose brain map prepared in advance; a cluster generation circuitry which generates a cluster by clustering the initial region of interest based on a myelin value included in the myelin feature image; and a segmentation circuitry which segments a personalized primary auditory cortex region by detecting and correcting an edge of the cluster. The primary auditory cortex region responsible for hearing is segmented, and high individual variability regions can be personalized.

### CROSS REFERENCE TO RELATED APPLICATIONS

This application claims priority to Korean Patent Application No. 10-2024-0141648, filed on Oct. 16, 2024, in the Korean Intellectual Property Office, which is incorporated by reference herein in its entirety.

### TECHNICAL FIELD

The present disclosure relates to a primary auditory cortex region segmenting device and method which are responsible for hearing, capable of segmenting the primary auditory cortex region in a personalized manner.

### BACKGROUND

Recently, technology using a magnetic resonance image (MRI) for obtaining an image for diagnosis and treatment in a medical treatment using a Nuclear Magnetic Resonance (NMR) phenomenon obtains an image having high contrast with respect to an organ or tissue inside a human body, and exerts power in diagnosing cancer, a tumor, and various diseases. In addition, the MRI is non-invasive to the human body and has advantages such as no damage from radiation unlike X-ray and CT devices. Accordingly, its demand has been increasing in recent years.

In particular, the human brain is related to many diseases, and in order to prevent such diseases, MRI imaging of the brain is required to obtain accurate information or to diagnose or treat already manifested brain diseases. The MRI image refers to an image obtained by measuring electromagnetic waves reflected after transmitting radio waves to a part of a human body in a strong magnetic field.

The ventricle, on the other hand, refers to the space within the human brain and is enclosed by the ependymal lining of the ventricles. The inside of the ventricles is filled with a liquid called cerebrospinal fluid (CSF), and the cerebrospinal fluid circulates in the ventricular system as a certain amount is produced and decomposed every day.

In the human brain, two left and right ventricles, a third ventricle and a fourth ventricle constitute the ventricle system, and the two ventricles in the left and right cerebral hemispheres are called lateral ventricles. One of the brain tissues, the lateral ventricle, can also be checked with brain MRI images, and information on the objective volume and size of the lateral ventricle can be obtained through automated techniques.

On the other hand, compartmentalization of structurally and functionally distinct regions in the human cerebral cortex is one of the main objectives of neuroscience.

In particular, one of the objectives is related to the compartmentalization of the primary auditory cortex region responsible for hearing. Although various methodologies have been proposed, it is very difficult to accurately define the boundary of the human primary auditory cortex at the individual level.

This is because the structure of the Heschl's Gyrus (HG) in which the auditory cortex is located is complex, and the shape, size, and location of the Heschl's Gyrus show great variability between hemispheres and individuals.

Therefore, there is a need for a personalized method for segmenting the primary auditory cortex area with high individual diversity.

(Patent Document 1) Korean Patent Published Patent Application No. 10-2018-0002234

### SUMMARY

The present disclosure has been made in an effort to provide a primary auditory cortex region segmenting device and method, which are capable of segmenting a primary auditory cortex region in charge of hearing and segmenting a primary auditory cortex region having great individual diversity into personalized regions.

According to an aspect of the present disclosure, there is provided a primary auditory cortex region segmenting device for segmenting a primary auditory cortical region in charge of hearing, the primary auditory cortical region segmentation device including an acquisition unit configured to collect multimodal magnetic resonance image data and obtain a myelin feature image and a curvature feature image from the multimodal magnetic resonance image data, a region of interest setting unit configured to set an initial region of interest for segmenting the auditory cortical region in the curvature feature image based on a pre-provided general brain map, a cluster generation unit configured to generate a cluster by clustering the initial region of interest based on a myelin value included in the myelin feature image, and a segmentation unit configured to detect and correct an edge of the cluster to segment a personalized primary auditory cortical region.

The multimodal magnetic resonance image data may include T1-weighted magnetic resonance image data and T2-weighted magnetic resonance image data, and the acquisition unit may obtain the myelin feature image by calculating a myelin value according to a brightness value ratio between the T1-weighted magnetic resonance image data and the T2-weighted magnetic resonance image data, and obtain the curvature feature image by calculating a curvature from the T1-weighted magnetic resonance image data.

The cluster generation unit may compare the myelin value in the initial region of interest with a threshold value provided in advance, generate a region in which the myelin value is less than or equal to the threshold value as a first cluster, and generate a region in which the myelin value exceeds the threshold value as a second cluster that is a candidate region of the personalized primary auditory cortex region.

The segmentation unit may extract a brain sulcus boundary within the initial region of interest from a curvature value included in the curvature feature image.

The segmentation unit may be configured to select the second cluster within the initial region of interest, and detect an edge of the second cluster based on the brain sulcus boundary.

The segmentation unit may be configured to adjust the edge of the second cluster by extending the edge to Heschl's gyrus, and to segment the personalized primary auditory cortex region based on the adjusted edge of the second cluster.

The segmentation unit may be configured to segment the edge of the second cluster corrected by extending a boundary of the gyrus into the personalized primary auditory cortex region.

The initial region of interest is a region for segmenting an auditory cortical region, and the general purpose brain map is an atlas brain map.

According to an aspect of another exemplary embodiment, there is provided a primary auditory cortex region segmenting method in a primary auditory cortex region segmenting device for segmenting a primary auditory cortex in charge of hearing, the method including: collecting multimodal magnetic resonance image data, and obtaining a myelin feature image and a curvature feature image from the multimodal magnetic resonance image data; setting an initial region of interest for segmenting the auditory cortex in the curvature feature image based on a pre-provided general-purpose brain map; clustering the initial region of interest based on a myelin value included in the myelin feature image to generate a cluster; and detecting and correcting an edge of the cluster to segment a personalized primary auditory cortex region.

According to one aspect of the present disclosure described above, the primary auditory cortex region segmenting device and method are provided, thereby segmenting the primary auditory cortex region responsible for hearing, and segmenting the primary auditory cortex region having great individual diversity to be personalized.

### BRIEF DESCRIPTION OF THE DRAWINGS

FIG. 1 is a view for explaining a configuration of a primary auditory cortex region segmenting device according to an embodiment of the present disclosure.

FIGS. 2 and 3 are views for explaining multimodal magnetic resonance image data collected by the primary auditory cortex region segmenting device according to an embodiment of the present disclosure

FIG. 4 is a view for explaining a myelin feature image acquired by the primary auditory cortex region segmenting device according to an embodiment of the present disclosure.

FIG. 5 is a view for explaining a curvature feature image acquired by the primary auditory cortex region segmenting device according to an embodiment of the present disclosure.

FIG. 6 is a view for explaining an initial region of interest set by the primary auditory cortex region segmenting device according to an embodiment of the present disclosure.

FIGS. 7 and 8 are diagrams for describing a cluster generated by a primary auditory cortex region segmenting device according to an embodiment of the present disclosure.

FIG. 9 is an exemplary diagram for describing a sulcus and a gyrus of a brain.

FIG. 10 is a diagram for describing a result of extracting a boundary of a sulcus of a brain by a primary auditory cortex region segmenting device according to an embodiment of the present disclosure.

FIG. 11 is a diagram for describing a result of detecting an edge of a second cluster by a primary auditory cortex region segmenting device according to an embodiment of the present disclosure.

FIG. 12 is a diagram for describing a result of correcting an edge of a second cluster by a primary auditory cortex region segmenting device according to an embodiment of the present disclosure.

FIG. 13 is a diagram for describing a personalized primary auditory cortex segmented by a primary auditory cortex region segmenting device according to an embodiment of the present disclosure.

FIGS. 14 and 15 are diagrams for describing a result of segmenting a personalized primary auditory cortex through a primary auditory cortex region segmenting device according to an embodiment of the present disclosure.

FIG. 16 is a flowchart for describing a primary auditory cortex region segmenting method according to an embodiment of the present disclosure.

In one or more implementations, not all the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

A detailed description of the present disclosure, which will be described later, refers to the accompanying drawings, which illustrate specific embodiments in which the present disclosure may be practiced as examples. These examples are described in detail to be sufficient for those skilled in the art to practice the present disclosure. It should be understood that the various embodiments of the present disclosure are different from each other but need not be mutually exclusive. For example, certain shapes, structures, and characteristics described herein may be implemented in other embodiments without departing from the spirit and scope of the present disclosure with respect to one embodiment. It should also be understood that the position or arrangement of individual components within each disclosed embodiment may be altered without departing from the spirit and scope of the present disclosure. Accordingly, the detailed description to be described below is not intended to be taken in a limited sense, and the scope of the present disclosure, if properly described, is limited only by the appended claims along with all the scope equivalent to those claimed by the claims. Similar reference numerals in the drawings refer to the same or similar functions across several aspects.

The components according to the present disclosure are components defined by functional classification rather than physical classification, and may be defined by functions performed by each. Each component may be implemented as hardware or a program code and a processing unit that perform each function, and functions of two or more components may be included in one component to be implemented. Accordingly, it should be noted that the names given to the components in the following embodiments are not intended to physically distinguish each component, but are given to imply a representative function in which each component is performed, and the technical spirit of the present disclosure is not limited by the names of the components.

Functions related to artificial intelligence according to the present disclosure are operated through a processor and a memory. The processor may be composed of one or a plurality of processors. In this case, the one or the plurality of processors may be general-purpose processors such as CPU, AP, and digital signal processor (DSP), graphic-only processors such as GPU and vision processing unit (VPU), or artificial intelligence-only processors such as NPU. The one or more processors control to process the input data according to a predefined operation rule or an artificial intelligence model stored in the memory. Alternatively, when one or a plurality of processors are dedicated artificial intelligence processors, the dedicated artificial intelligence processor may be designed in a hardware structure specialized for processing a specific artificial intelligence model.

The predefined motion rule or artificial intelligence model is characterized in that it is made through learning. Here, being created through learning means that a basic artificial intelligence model is trained using a plurality of learning data by a learning algorithm, thereby creating a predefined operation rule or an artificial intelligence model set to perform a desired characteristic (or purpose). Such learning may be performed in the device itself in which the artificial intelligence according to the present disclosure is performed, or may be performed through a separate server and/or system. Examples of the learning algorithm include supervised learning, unsupervised learning, semi-supervised learning, or reinforcement learning, but are not limited to the above-described examples.

The artificial intelligence model may be composed of a plurality of neural network layers. Each of the plurality of neural network layers has a plurality of weight values, and a neural network operation is performed through an operation between an operation result of a previous layer and a plurality of weights. A plurality of weights of the plurality of neural network layers may be optimized by a learning result of the artificial intelligence model. For example, a plurality of weights may be updated such that a loss value or a cost value obtained from the artificial intelligence model is reduced or minimized during the learning process. The artificial neural network may include a deep neural network (DNN), for example, a Convolutional Neural Network (CNN), a Deep Neural Network (DNN), a Recurrent Neural Network (RNN), a Restricted Boltzmann Machine (RBM), a Deep Barrier Network (DBN), a Bidirectional Recurrent Deep Neural Network (BRDNN), or a deep Q-network (Deep Q-Networks), but is not limited thereto.

According to an exemplary embodiment of the present disclosure, the processor may implement artificial intelligence. Artificial intelligence refers to an artificial neural network-based machine learning method that simulates human neural cells and allows machines to learn. The artificial intelligence methodology may be segmented into supervised learning in which an answer (output data) of a problem (input data) is determined by providing input data and output data together as training data according to a training method, unsupervised learning in which an answer (output data) of the problem (input data) is not determined by providing only input data without output data, and reinforcement learning in which learning is performed in a direction that maximizes such compensation as a reward is given in an external environment whenever an action is taken in a current state. In addition, the AI methodology may be classified according to an architecture that is a structure of a learning model, and the widely used architecture of deep learning technology may be classified into a CNN (Convolutional Neural Network), a RNN (Recurrent Neural Network), a transformer, a GAN (generative adversarial networks), and the like.

The device and the system may include an artificial intelligence model. The artificial intelligence model may be one artificial intelligence model, or may be implemented as a plurality of artificial intelligence models. The artificial intelligence model may be composed of a neural network (or an artificial neural network), and may include a statistical learning algorithm that mimics nerves of biology in machine learning and cognitive science. The neural network may refer to the entire model having problem-solving ability by changing the strength of the synaptic coupling through learning by an artificial neuron (node) that has formed a network by combining synapses. The neurons of the neural network may include a combination of weights or biases. The neural network may include one or more layers composed of one or more neurons or nodes. In an embodiment, the device may include input layer, hidden layer, and output layer. The neural network constituting the device may infer an output to be predicted from an arbitrary input by changing the weight of the neuron through learning.

The processor may generate a neural network, train the neural network, perform an operation based on received input data, generate an information signal based on a result of the operation, or retrain the neural network. Models of the neural network may include various types of models such as a Convolution Neural Network (CNN) such as GoogleNet, AlexNet, VGG Network, and the like, a Region with Convolution Neural Network (R-CNN), a Region Proposal Network (RPN), a Recurrent Neural Network (RNN), a Tracking-based deep neural network (S-DNN), a State-Space Dynamic Neural Network (S-SDNN), a Deconvolution Network, a Deep Barrier Network (DBN), a Structured Boltzman Machine (RBM), a Fully Convolutional Network, a Long Short-Term Memory (LSTM) Network, Classification Network, and the like, but are not limited thereto. The processor may include one or more processors for performing operations according to models of the neural network. For example, the neural network may include a deep neural network.

Neural networks include Convolutional Neural Network (CNN), Recurrent Neural Network (RNN), Perceptron, Multilayer Perceptron, Feed Forward (FF), Radial Basis Network (RBF), Deep Feed Forward (DFF), Long Short Term Memory (LSTM), Gated Recurrent Unit (GRU), Auto Encoder (AE), Variable Automatic Encoder (VAE), Denoising Auto Encoder (DAE), and Denato Decoder (SAE). Markov Chain, Handfield Network (HN), Boltzmann Machine (BM), Restricted Boltzmann Machine (MC), Depp Barrier Network (RBM), Deep Convolutional Network (DBN), Deep Voltage Inverse Graphics Network (DCN), Generic Advertised Network (GAN), Liquid State Machine (DCIGN), Extreme Differential Network (GAN), Extreme Feed Network (LSM), Extreme Feed Network (ELM), Extreme Feed Network (ESN) A neural computer, a neural turning machine (DRN), a capsule network (DNC), a Korean network (NTM), and an attachment network (CN KN AN) may be included, but it will be understood by those skilled in the art that any neural network may be included without being limited thereto.

According to an exemplary embodiment of the present disclosure, a processor may be configured to perform a convolutional neural network (CNN) such as GoogleNet, AlexNet, VGG Network, or the like, a region with convolutional neural network (R-CNN), a region professional network (RPN), a recurrent neural network (RNN), a standing-based deep neural network (S-DNN), a state-space dynamic neural network (S-SDNN), a Deconvolution Network, a deep fundamental network (DBN), a structured short circuit network (RBM), a Fully Convolutional Network, a LSTM (Long Short-Term Memory) Network, Classification Network, Generative Modeling, eXplainable AI, Continual AI, Representation Learning, AI for Material Design, BERT, SP-BERT, MRC/QA, Text Analysis, Dialog System, GPT-3, GPT-4, Visual Analytics, Visual Understanding, and the like, and a natural language processing Various artificial intelligence structures and algorithms such as Video Synthesis, ResNet, Anomaly Detection, Prediction, Time-Series Forecasting for vision processing, Optimization, Recommendation, Data Creation for data intelligence, etc., may be used, but are not limited thereto.

Hereinafter, exemplary embodiments of the present disclosure will be described in more detail with reference to the drawings.

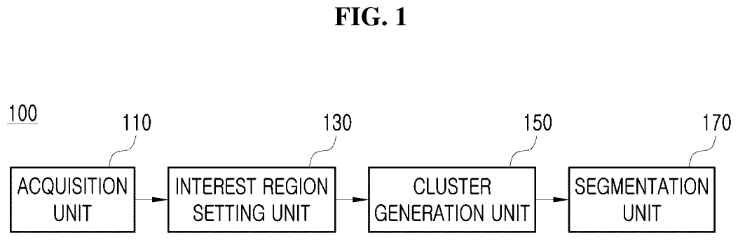

FIG. 1 is a diagram for describing a configuration of a primary auditory cortex region segmenting device 100 according to an embodiment of the present disclosure.

The primary auditory cortex region segmenting device 100 according to the present embodiment is provided to segment a primary auditory cortex region having a large personal diversity into a personalized form.

Specifically, the auditory cortex region has a different shape for each individual in the region responsible for language unlike other regions.

The most commonly used technique among conventional techniques for separating, i.e., segmenting, brain regions is a statistical techniques such as manual anatomical delineation or clustering methods.

Group modeling is possible in areas other than the auditory cortex region because they are similar, whereas the auditory cortex region is so different from person to person that personalized region segmentation is required.

Accordingly, in order to segment the personalized auditory cortex region, the device 100 according to the present embodiment may include the acquisition unit 110 (e.g., acquisition circuitry), the region of interest setting unit 130 (e.g., region of interest setting circuitry), the cluster generation unit 150 (e.g., cluster generation circuitry), and the segmentation unit 170 (e.g., segmentation circuitry), and may segment a specific region of a small size rather than a large brain tissue from a personalized perspective by applying a machine learning technique to brain magnetic resonance image data.

In addition, the device 100 may be executed by installing software (applications) for performing the primary auditory cortex region segmenting method, and the acquisition unit 110, the region of interest setting unit 130, the cluster generation unit 150, and the segmentation unit 170 may be controlled by software (applications) for performing the primary auditory cortical region segmentation method.

In this case, the device 100 may be a separate terminal or some module of the terminal. In addition, the acquisition unit 110, the region of interest setting unit 130, the cluster generation unit 150, and the segmentation unit 170 may be formed as an integrated module or may be formed as one or more modules. However, on the contrary, each configuration may be formed as a separate module.

In addition, the device 100 may have mobility or may be fixed. The device 100 may be in the form of a server or an engine, and may be referred to as other terms such as a device, an appliance, a terminal, a user equipment (UE), a mobile station (MS), a wireless device, and a handheld device. The device 100 may execute or manufacture various software based on an Operation System (OS), that is, a system. Here, the operating system is a system program for enabling software to use hardware of a device, and may include all of mobile computer operating systems such as Android OS, iOS, Windows mobile OS, Bada OS, Symbian OS, and BlackBerry OS, and computer operating systems such as Windows, Linux, Unix, MAC, AIX, and HP-UX.

In addition, the device 100 may include a storage unit in which a computer program for performing the primary auditory cortex region segmenting method is recorded.

First, the acquisition unit 110 according to the present embodiment may collect multimodal magnetic resonance image data.

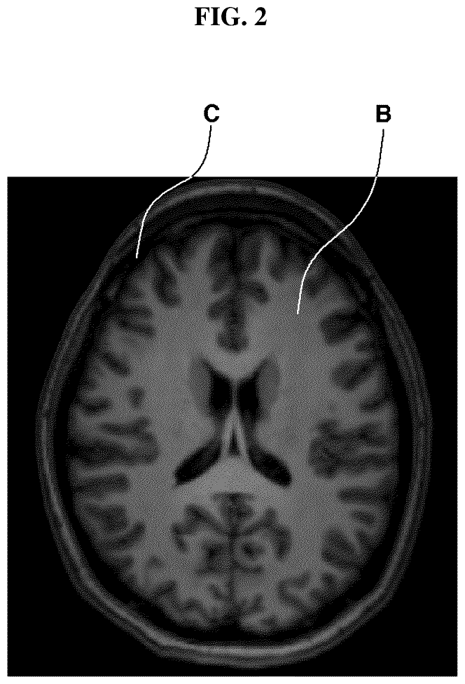

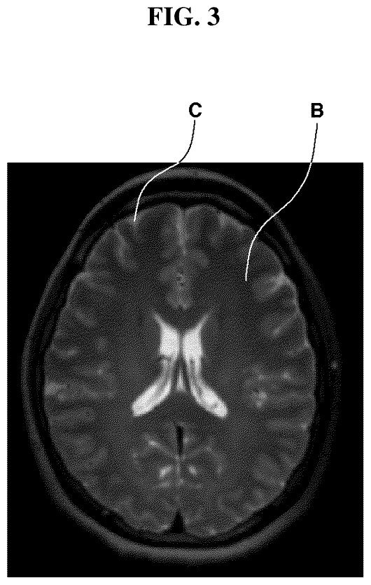

FIGS. 2 and 3 are diagrams illustrating multimodal magnetic resonance image data collected by the acquisition unit 110 according to an embodiment of the present disclosure.

The multi-modal magnetic resonance image data collected by the acquisition unit 110 according to the present embodiment may include T1-weighted (e.g., longitudinal relaxation time weighted) magnetic resonance image data and T2-weighted (e.g., transverse relaxation time weighted) magnetic resonance image data.

The T1-weighted magnetic resonance image data may mean data in which the inside of the brain B is bright and the outer cortex C is dark as shown in FIG. 2, whereas the T2-weighted magnetic resonance image data may mean data in which the cortex region C is bright and the inside B is dark as opposed to the T1-weighted magnetic resonance image data as shown in FIG. 3.

Meanwhile, the acquisition unit 110 according to the present embodiment may acquire a myelin feature image and a curvature feature image from the multimodal magnetic resonance image data in which these different regions are emphasized.

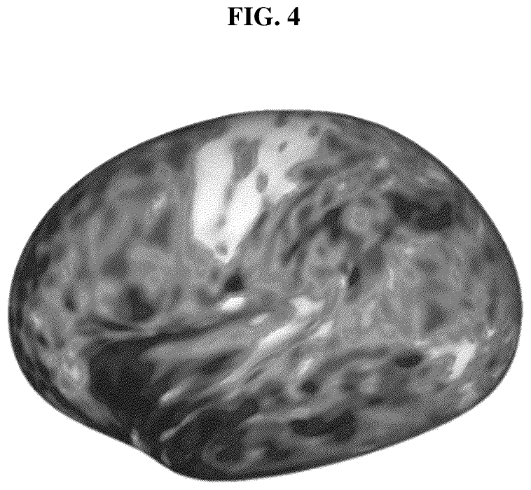

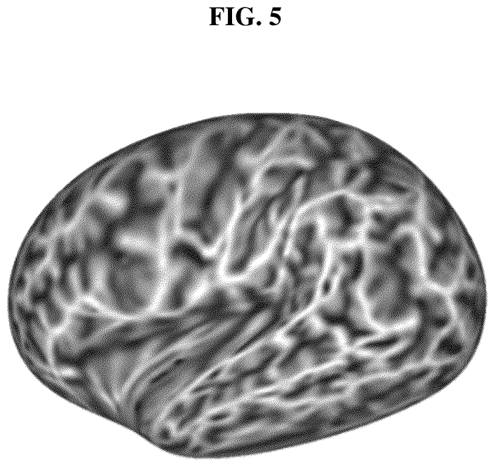

FIG. 4 is a diagram for describing a myelin feature image acquired by the acquisition unit 110, and FIG. 5 is a diagram for describing a curvature feature image acquired by the acquisition unit 110.

The acquisition unit 110 may acquire a myelin feature image as shown in FIG. 4 by calculating a myelin value according to a brightness value ratio between the T1-weighted magnetic resonance image data and the T2-weighted magnetic resonance image data.

In addition, the acquisition unit 110 may acquire a curvature feature image as shown in FIG. 5 by calculating a curvature from the T1-weighted magnetic resonance image data.

Meanwhile, the region of interest setting unit 130 according to the present embodiment may set an initial region of interest in the curvature feature image acquired by the acquisition unit 110.

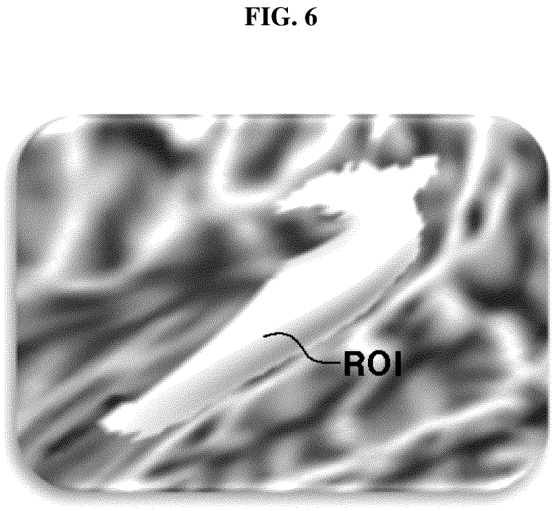

FIG. 6 is an exemplary diagram of an initial region of interest set by the region of interest setting unit 130 according to the present embodiment.

As illustrated in FIG. 6, the region of interest setting unit 130 sets an initial region of interest ROI in the curvature feature image based on a general brain map provided in advance, and the initial region of interest is a region for segmenting an auditory cortical region, and may mean a region expected to be a primary auditory cortex. In addition, the general-purpose brain map may be various known types of brain maps, for example, an atlas brain map.

Meanwhile, the cluster generation unit 150 is provided to generate a cluster from the initial region of interest.

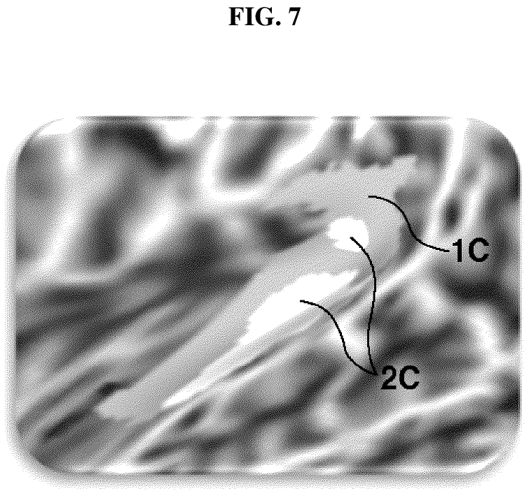

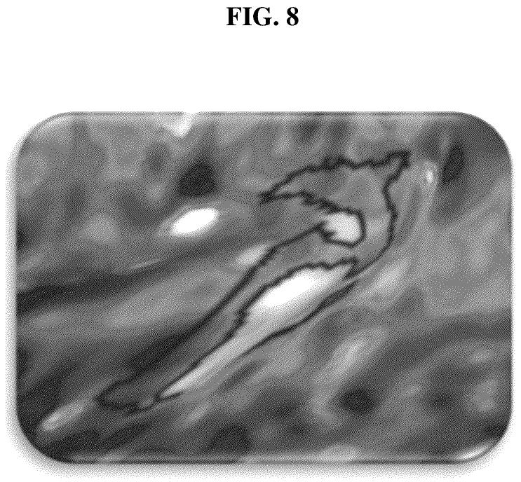

FIGS. 7 and 8 are exemplary diagrams for explaining a cluster generated by the cluster generation unit 150.

The cluster generation unit 150 according to the present embodiment may generate a cluster by clustering the initial region of interest based on the myelin value included in the myelin feature image.

To this end, the cluster generation unit 150 may apply a Gaussian mixed model clustering technique to the myelin value, and in detail, the cluster generation unit 150 may compare the myelin value in the initial region of interest with a threshold value provided in advance to generate the cluster.

In addition, the cluster generation unit 150 may generate a region in which the myelin value is less than or equal to the threshold value as the first cluster, and generate a region in which the myelin value exceeds the threshold value as the second cluster.

In this case, the second cluster generated by the cluster generation unit 150 may be a candidate region of the personalized primary auditory cortex region.

FIG. 7 is a diagram illustrating a first cluster 1C and a second cluster 2C generated by the cluster generation unit 150 with a curvature feature image as a background.

Meanwhile, the segmentation unit 170 is provided to segment the personalized primary auditory cortex region by detecting and correcting the edge of the cluster generated by the cluster generation unit 150.

To this end, the segmentation unit 170 may match the curvature value to the cluster generated by the cluster generation unit 150.

In addition, the segmentation unit 170 may detect edges of the first cluster and the second cluster generated by the cluster generation unit 150, and may be illustrated as a background of the myelin image as shown in FIG. 8.

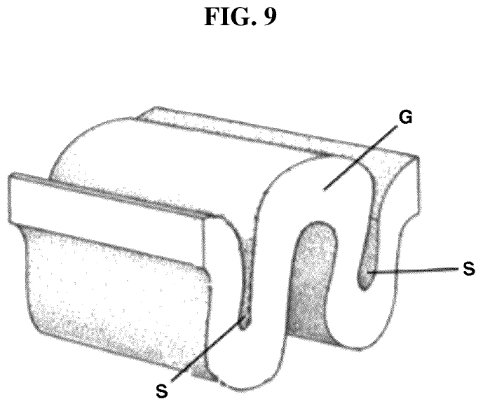

FIG. 9 is an exemplary view for explaining the sulcus S and gyrus G of the brain, and as shown in FIG. 9, there are sulcus and gyrus in the brain.

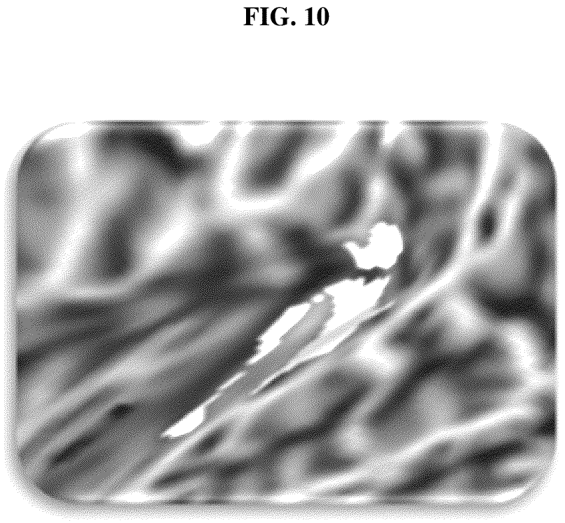

Accordingly, the segmentation unit 170 according to the present embodiment may extract the boundary of the brain sulcus in the initial region of interest from the curvature value included in the curvature feature image, and FIG. 10 is an exemplary diagram of a result of the segmentation unit 170 extracting the boundary of the brain sulcus in the initial region of interest.

The segmentation unit 170 according to the present embodiment may extract a brain sulcus in the initial region of interest in order to further widen the sulcus.

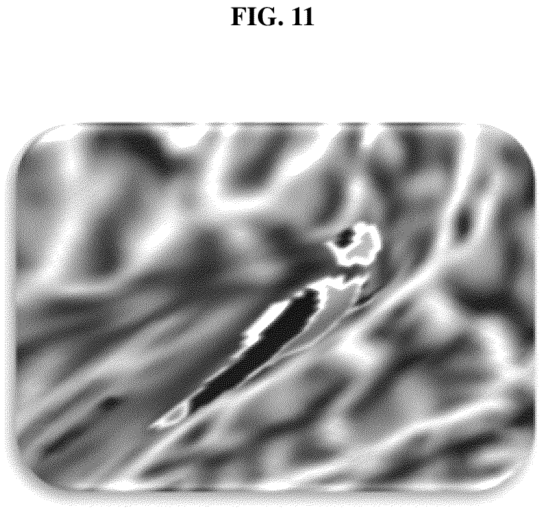

In addition, the segmentation unit 170 according to the present embodiment may select a region corresponding to the second cluster in the initial region of interest and detect an edge of the second cluster based on the extracted brain sulcus boundary. FIG. 11 is an exemplary diagram of a result of detecting, by the segmentation unit 170, an edge of a second cluster based on a brain sulcus boundary. That is, the segmentation unit 170 according to the present embodiment may cut the group obtained based on the myelin value into the brain sulcus defined based on the curvature value.

In addition, the segmentation unit 170 may correct the edge of the second cluster by extending the detected edge of the second cluster to Heschl's gyrus as shown in FIG. 11, and may segment the individualized primary auditory cortex region based on the corrected edge of the second cluster.

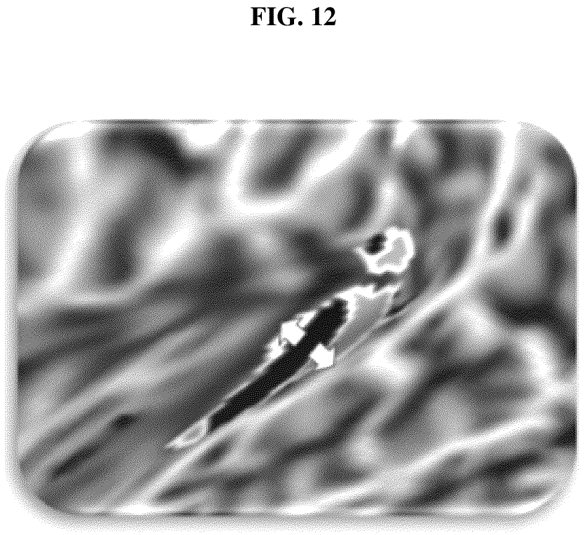

FIG. 12 is an exemplary diagram of a result of correcting by expanding the edge of the second cluster to the Heschl's gyrus by the segmentation unit 170 of the present disclosure.

Correcting the edge of the second cluster by the segmentation unit 170 may mean expanding the edge until the brain sulcus reaches the boundary with the gyrus based on the curvature.

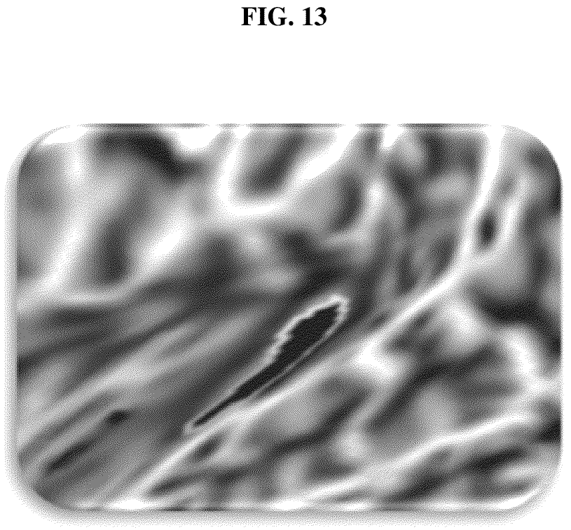

The segmentation unit 170 may segment the edge of the second cluster corrected by extending to the boundary of the gyrus into a personalized primary auditory cortex region, and FIG. 13 is an exemplary diagram of the personalized primary auditory cortex region segmented by the segmentation unit 170.

The device 100 according to the present embodiment may segment the primary auditory cortex region into a personalized primary auditory cortex region, which is not formed only as a single region, as well as having different positions for each individual through the above process.

The device 100 may be integrated into the MRI device or provided separately from the MRI device, and may be based on an artificial intelligence model trained to output a myelin feature image and a curvature feature image by calculating a myelin value or calculating a curvature value when magnetic resonance image data is input.

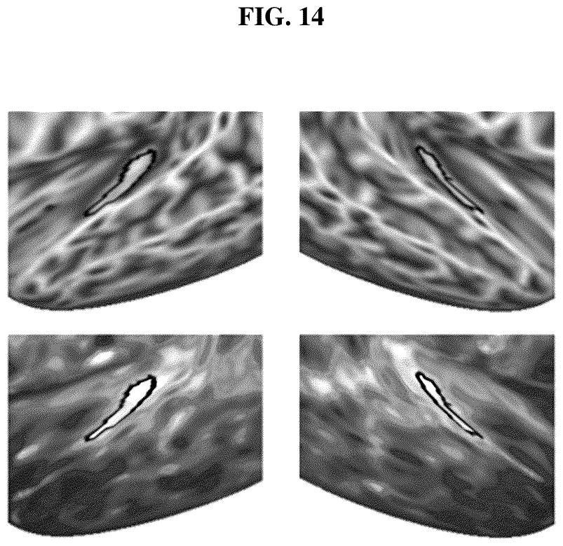

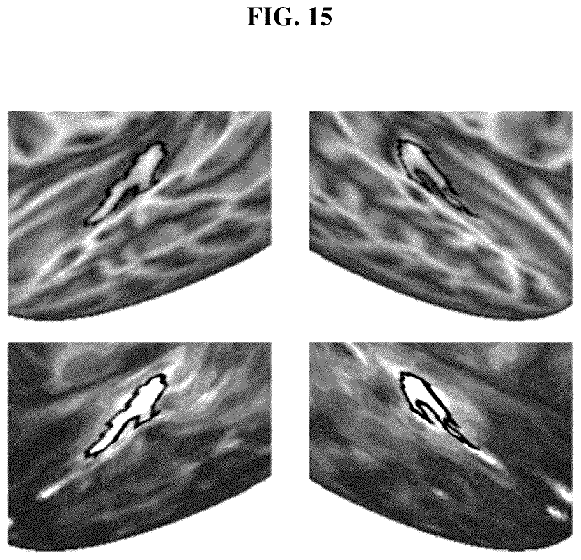

FIGS. 14 and 15 are diagrams illustrating a result of segmenting a personalized primary auditory cortex region through the device 100 according to an embodiment of the present disclosure, in which the black-and-white images of FIGS. 14 and 15 are diagrams illustrating the personalized primary auditory cortex region as a background of a curvature feature image, and the color image is a diagram illustrating the personalized primary auditory cortex region as a background of a myelin feature image.

Specifically, FIGS. 14 and 15 are results obtained by segmenting personalized primary auditory cortex regions from multimodal magnetic resonance image data of different people.

The device 100 according to the present embodiment may individually segment the primary auditory cortex region of an individual in which the primary auditory cortex region is one piece as shown in FIG. 14 and an individual in which the primary auditory cortex region is segmented into two pieces as shown in FIG. 15.

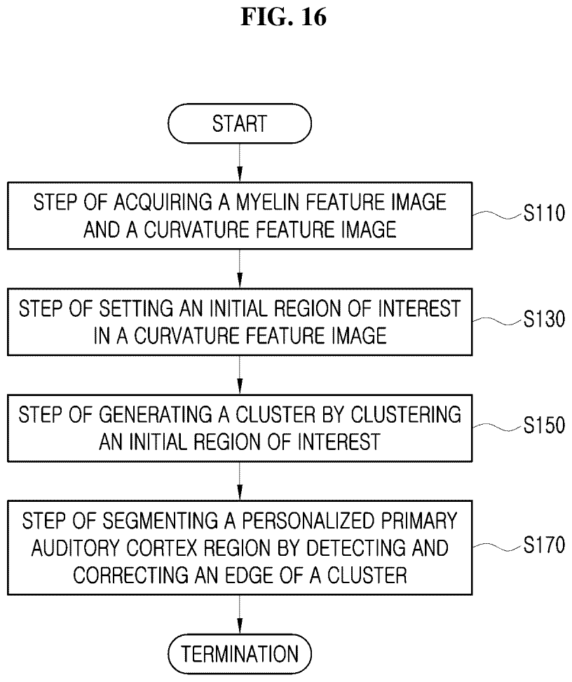

FIG. 16 is a flowchart illustrating a primary auditory cortex region segmenting method according to an embodiment of the present disclosure, and since the primary auditory cortex region segmenting method according to an embodiment of the present disclosure is performed on substantially the same configuration as the device 100 shown in FIG. 1, the same reference numerals are assigned to the same components as the device 100 of FIG. 1, and repeated descriptions thereof will be omitted.

The primary auditory cortex region segmenting method according to the present embodiment includes obtaining a myelin feature image and a curvature feature image (S110), setting an initial region of interest in the curvature feature image (S130), clustering the initial region of interest to generate a cluster (S150), and segmenting a personalized primary auditory cortex region by detecting and correcting an edge of the cluster (S170).

In the acquiring of the myelin feature image and the curvature feature image (S110), the acquiring unit 110 may collect multimodal magnetic resonance image data and acquire the myelin feature image and the curvature feature image from the multimodal magnetic resonance image data.

In the step S130 of setting the initial region of interest in the curvature feature image, the region of interest setting unit 130 may set the initial region of interest for the auditory cortical region segmentation in the curvature feature image based on a general-purpose brain map prepared in advance.

Meanwhile, in the step S150 of generating the cluster by clustering the initial region of interest (ROI), the cluster generation unit 150 may generate the cluster by clustering the initial ROI based on the myelin value included in the myelin feature image.

In the step S170 of segmenting the personalized primary auditory cortex region by detecting and correcting the edge of the cluster, the segmentation unit 170 may detect the edge of the cluster generated in the step S150 of generating the cluster, and correct the detected edge, thereby segmenting the personalized primary auditory cortex region according to the corrected edge.

The primary auditory cortex region segmenting method of the present disclosure may be implemented in the form of program instructions that may be executed through various computer components and recorded in a computer-readable recording medium. The computer-readable recording medium may include program instructions, data files, data structures, and the like alone or in combination.

The program instructions recorded in the computer-readable recording medium may be specially designed and configured for the present disclosure or may be known to and used by those skilled in the field of computer software.

Examples of the computer-readable recording medium include a magnetic medium such as a hard disk, a floppy disk, and a magnetic tape, an optical recording medium such as a CD-ROM and a DVD, a magneto-optical medium such as a floptical disk, and a hardware device specially configured to store and execute program instructions such as a ROM, a RAM, a flash memory, and the like.

Examples of program instructions include not only machine language codes such as those generated by a compiler, but also high-level language codes that may be executed by a computer using an interpreter or the like. The hardware device may be configured to operate as one or more software modules to perform processing according to the present disclosure, and vice versa.

Although various embodiments of the present disclosure have been illustrated and described above, the present disclosure is not limited to the specific embodiments described above, and various modifications can be made by a person skilled in the art to which the present disclosure belongs without departing from the gist of the present disclosure claimed in the claims, and such modifications should not be individually understood from the technical spirit or the prospect of the present disclosure.

What is claimed is:

1. A primary auditory cortex region segmenting device for segmenting a primary auditory cortical region in charge of hearing, the primary auditory cortex region segmenting device comprising:
acquisition circuitry configured to collect multimodal magnetic resonance image data, and to obtain a myelin feature image and a curvature feature image from the multimodal magnetic resonance image data;
region of interest setting circuitry configured to set an initial region of interest for segmenting the auditory cortical region in the curvature feature image based on a general-purpose brain map prepared in advance;
cluster generation circuitry configured to cluster the initial region of interest based on a myelin value included in the myelin feature image to generate a cluster; and
segmentation circuitry configured to detect and correct an edge of the cluster to segment a personalized primary auditory cortical region.

2. The primary auditory cortex region segmenting device of claim 1, wherein the multimodal magnetic resonance image data comprises T1-weighted magnetic resonance image data and T2-weighted magnetic resonance image data, and wherein the acquisition circuitry obtains the myelin feature image by calculating a myelin value according to a brightness value ratio between the T1-weighted magnetic resonance image data and the T2-weighted magnetic resonance image data, and obtains the curvature feature image by calculating a curvature from the T1-weighted magnetic resonance image data.

3. The primary auditory cortex region segmenting device of claim 2, wherein the cluster generation circuitry compares the myelin value in the initial region of interest with a threshold value provided in advance, generates a region in which the myelin value is less than or equal to the threshold value as a first cluster, and generates a region in which the myelin value exceeds the threshold value as a second cluster that is a candidate region of the personalized primary auditory cortex region.

4. The primary auditory cortex region segmenting device of claim 3, wherein the segmentation circuitry extracts a brain sulcus boundary within the initial region of interest from a curvature value included in the curvature feature image.

5. The primary auditory cortex region segmenting device of claim 4, wherein the segmentation circuitry selects the second cluster within the initial region of interest and detects an edge of the second cluster based on the brain sulcus boundary.

6. The primary auditory cortex region segmenting device of claim 5, wherein the segmentation circuitry is further configured to correct the edge of the second cluster by extending it to a Heschl's gyrus, and to segment the personalized primary auditory cortex region based on the corrected edge.

7. The primary auditory cortex region segmenting device of claim 6, wherein the segmentation circuitry is further configured to segment the edge of the second cluster corrected by extending a boundary of the gyrus into the personalized primary auditory cortex region.

8. The primary auditory cortex region segmenting device of claim 1, wherein the initial region of interest is a region for segmenting an auditory cortical region, and the general purpose brain map is an atlas brain map.

9. A primary auditory cortex region segmenting method in a primary auditory cortex region segmenting device for segmenting a primary auditory cortical region responsible for hearing, the method comprising:
collecting multimodal magnetic resonance image data and obtaining a myelin feature image and a curvature feature image from the multimodal magnetic resonance image data;
setting an initial region of interest for segmenting the auditory cortical region in the curvature feature image based on a pre-prepared general-purpose brain map;
clustering the initial region of interest based on a myelin value included in the myelin feature image to generate a cluster; and
detecting and correcting an edge of the cluster to segment a personalized primary auditory cortical region.

10. The method of claim 9, wherein the multimodal magnetic resonance image data comprises T1-weighted magnetic resonance image data and T2-weighted magnetic resonance image data, and wherein the obtaining the myelin feature image comprises calculating a myelin value according to a brightness value ratio between the T1-weighted magnetic resonance image data and the T2-weighted magnetic resonance image data, and the obtaining the curvature feature image comprises calculating a curvature from the T1-weighted magnetic resonance image data.

11. The method of claim 10, wherein the method further comprises:
comparing the myelin value in the initial region of interest with a threshold value provided in advance;
generating a region in which the myelin value is less than or equal to the threshold value as a first cluster; and
generating a region in which the myelin value exceeds the threshold value as a second cluster that is a candidate region of the personalized primary auditory cortex region.

12. The method of claim 11, wherein the detecting and correcting the edge further comprises:
extracting a brain sulcus boundary within the initial region of interest from a curvature value included in the curvature feature image.

13. The method of claim 12, further comprising:
selecting the second cluster within the initial region of interest; and
detecting an edge of the second cluster based on the brain sulcus boundary.

14. The method of claim 13, further comprising:
correcting the edge of the second cluster by extending it to a Heschl's gyrus, and to segment the personalized primary auditory cortex region based on the corrected edge.

15. The method of claim 14, further comprising:
segmenting the edge of the second cluster corrected by extending a boundary of the gyrus into the personalized primary auditory cortex region.

16. The method of claim 1, wherein the initial region of interest is a region for segmenting an auditory cortical region, and the general purpose brain map is an atlas brain map.
